(19) United States
(12) Patent Application Publication
(10) Pub. No.: US 2026/0123945 A1
(43) Pub. Date: May 7, 2026

(54) Histotripsy Apparatus With Acoustically Registered Auxiliary Probe

(71) Applicant: Wisconsin Alumni Research Foundation, Madison, WI (US)

(72) Inventor: Martin Gerd Wagner, Madison, WI (US)

(21) Appl. No.: 18/934,467

(22) Filed: Nov. 1, 2024

(51) Int. Cl. A61B 17/22; A61B 8/00; A61B 8/12

(52) U.S. Cl. CPC A61B 17/22004; A61B 8/12; A61B 8/4245; A61B 8/463; A61B 2017/22008; A61B 2017/22028

(57) ABSTRACT

A histotripsy device treating tissue with cavitating microbubbles produced by a high power therapy ultrasonic transducer provides an independently positionable ultrasound probe located and registered to the therapy ultrasonic transducer using ultrasonic ranging signals communicating through the patient. In one embodiment the independently positionable ultrasound probe is an intra-cavity probe.

### STATEMENT REGARDING FEDERALLY SPONSORED RESEARCH OR DEVELOPMENT

This invention was made with government support under EB031007 awarded by the National Institutes of Health. The government has certain rights in the invention.

### BACKGROUND OF THE INVENTION

The present invention relates generally to histotripsy devices using high intensity ultrasound to destroy targeted tissue and in particular to an auxiliary ultrasound imaging probe for a histotripsy device.

Histotripsy employs short, intense bursts of ultrasonic acoustic energy from a therapy transducer outside of a patient to generate microbubbles at a focal point within tissue. The rapid expansion and collapse of the microbubbles disrupts tissue cell walls destroying those cells while avoiding the side effects of more invasive treatments such as cryo-or thermal ablation.

The energy of a histotripsy device may be targeted using an imaging probe fixed with respect to the therapy transducer. While such a fixed imaging probe desirably maintains registration with the therapy transducer focal spot, it precludes repositioning the imaging probe for better coupling to patient tissue or to avoid obstructions such as bone or air cavities, or the use of intra-cavity probes whose position is not readily fixed.

### SUMMARY OF THE INVENTION

The present invention provides an independently positionable ultrasound probe for a histotripsy system whose image may be registered to the therapy transducer using acoustic signals transmitted through the patient. In some embodiments, these acoustic signals may be produced and detected using the existing therapy transducer and transducer elements of the ultrasound probe and thus may be implemented with minimal hardware modifications to existing histotripsy machines.

In one embodiment, the invention provides an ultrasound therapy system including an ultrasound therapy transducer array adapted to deliver histotripsy pulses into a subject to generate cavitation in the subject at a focal region. The invention further provides an ultrasound probe independently positionable with respect to the ultrasound therapy transducer array and adapted to receive acoustic signals from the subject for the production of an ultrasonic image of the subject. A first set of ultrasonic transducer elements is positioned at predetermined separated locations fixed with respect to the ultrasonic therapy transducer array and adapted to couple acoustic signals between the first set of ultrasonic transducer elements and the subject. A second set of ultrasonic transducer elements is positioned at predetermined separated locations fixed with respect to the ultrasound probe and adapted to couple acoustic signals between the second set of ultrasonic transducer elements and the subject. A localizing circuit operates to activate transducers of one of the first or second set of ultrasonic transducers to couple independently distinguishable locating acoustic signals into the subject and to monitor a receipt of the independently distinguishable locating acoustic signals at transducers of a different of the first and second set of ultrasonic transducers to deduce a relative location of the ultrasonic probe with respect to the ultrasound therapy transducer.

It is thus a feature of at least one embodiment of the invention to greatly improve the imaging of the treatment site in histotripsy by using an independently positionable ultrasonic probe that is nevertheless registered to the therapy transducer.

The second set of ultrasonic transducers may be separated in two dimensions and the localizing circuit may uniquely localize the ultrasonic probe with respect to relative translation and rotation of the ultrasonic probe with respect to the ultrasonic therapy transducer.

It is thus a feature of at least one embodiment of the invention to fully characterize the field-of-view of the independent ultrasonic probe to assist the user in orienting the obtained image with respect to the therapy transducer and/or previously obtained reference images of the patient.

The system may include at least one image processing circuit communicating with the ultrasound probe to produce an ultrasonic image of the subject from ultrasonic signals received by the ultrasound probe and a compositor communicating with the localizing circuit to register a fiducial mark representing the focal region with respect to the ultrasonic image. A display may communicate with the compositor to display an image of the subject registered to the fiducial mark. Alternatively or in addition, a targeting mark may be placed on the ultrasonic image and used to control the therapy transducer to move its focus into alignment with that targeting mark according to the registration.

It is thus a feature of at least one embodiment of the invention to use the registration of the ultrasonic probe to provide the user with an indication of the location of the therapy transducer focal spot in the ultrasonic image or to guide movement of the therapy transducer.

The ultrasound probe may transmit and receive ultrasonic signals for the production of the ultrasonic image of the subject.

It is thus a feature of at least one embodiment of the invention to allow use with a standard ultrasound probe, for example, as may produce backscatter images.

The system may include a computer memory holding non-ultrasound image data of the subject obtained from a non-ultrasonic imaging modality, and the compositor may operate to register the non-ultrasound image data to the ultrasonic image to display the same.

It is thus a feature of at least one object of the invention to allow fusion of real time microbubble imaging with patient data acquired through different modalities, for example highlighting tumors tissue.

The first set of ultrasonic transducer elements may provide at least three ultrasonic transducer elements.

It is thus a feature of at least one embodiment of the invention to permit complete characterization of the location of the ultrasound probe transducers for most treatment situations, for example, transthoracic histotripsy.

The first set of ultrasonic transducer elements may operate alternatively to provide the independently distinguishable locating acoustic signals and histotripsy pulses.

It is thus a feature of at least one embodiment of the invention to make use of existing hardware for the purpose of providing a registered, independent ultrasound probe. In this case the registration signals may be transmitted from the transducers producing histotripsy pulses.

The localizing circuit may deduce the relative location of the different receiving ultrasonic transducer elements based on a speed of sound through the subject between the first and second set of ultrasonic transducers and may further include a sound speed corrector iteratively correcting an assumed sound speed using the deduced relative location of the ultrasonic probe with respect to the ultrasonic therapy transducer.

It is thus a feature of at least one embodiment of the invention to address variations in sound speed (or loss of sound speed signal) possible when using sound transmitted through inhomogeneous tissue of the body for localization.

The sound speed corrector may iteratively correct an assumed sound speed using at least one of image data obtained from the ultrasound probe and image data obtained from a non-ultrasound imaging modality.

It is thus a feature of at least one embodiment of the invention to make use of the ultrasound probe to characterize the signal path as may affect changes in sound speed.

The ultrasound probe may be adapted for intra-cavity use with in the patient.

It is thus a feature of at least one embodiment of the invention to permit histotripsy using intra-cavity ultrasound probes whose positions cannot be readily fixed with respect to the therapy transducers.

These particular objects and advantages may apply to only some embodiments falling within the claims and thus do not define the scope of the invention.

### BRIEF DESCRIPTION OF THE DRAWINGS

FIG. 1 is a block diagram of a histotripsy apparatus incorporating the features of the present invention providing an independently locatable ultrasound probe such as an intra-cavity probe;

FIG. 2 is a diagram showing the geometric measurements for determining the location and orientation of an ultrasonic probe element deduced from distances from beacon transmitters fixed with respect to the histotripsy transducer; and

FIG. 3 is a flowchart showing the steps of data processing implemented by the system of FIG. 1.

### DETAILED DESCRIPTION OF THE PREFERRED EMBODIMENT

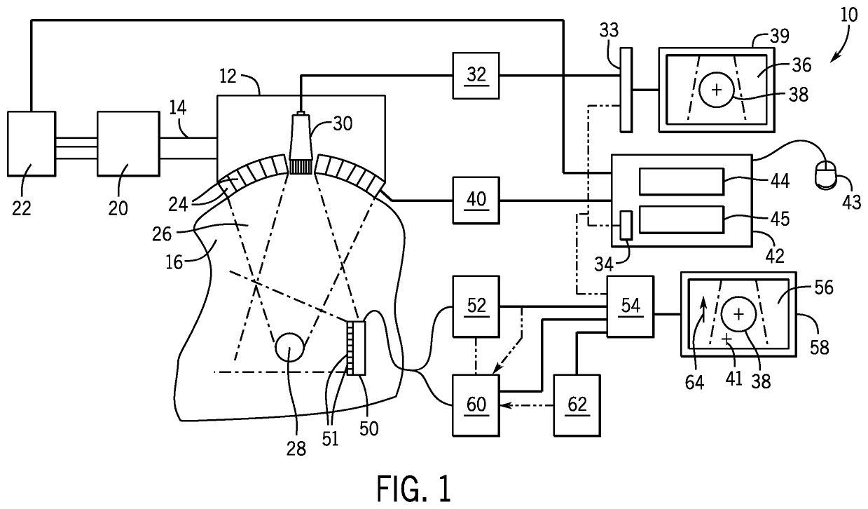

Referring now to FIG. 1, a histotripsy machine 10 may provide for a therapy transducer array 12 supported, for example, on an arm 14 that may be positioned with respect to a patient 16 by servo positioning motors 20 and servo driver electronics 22. A lower operating surface of the therapy transducer array 12, for example, as positioned for a transabdominal operation, may include an array of ultrasonic transducer elements 24 arranged to provide a focused beam of ultrasonic energy 26 converging at a focal point 28 to produce microbubble cavitation for the destruction of tumorous tissue.

An ultrasonic probe 30 may be directed downwardly in alignment with the ultrasonic energy 26 transmitted by the therapy transducer array 12 to independently transmit and receive ultrasonic signals for imaging a region around the focal point 28, thereby assisting in aligning the focal point 28 with the tissue to be treated and the monitoring of that tissue treatment. The ultrasound imaging probe 30 may communicate with image processing circuitry 32 for the development of a B-mode ultrasound image data provided to a compositor 33. The compositor 33 also receives focus data 34 indicating the position of the focal point 28 with respect to the therapy transducer array 12 so that the compositor 33 may present on display 39 a B-mode image 36 of the patient 16 with a fiducial mark 38 indicating a location of the focal point 28. It will be understood that the relative position of the focal point 28 may be determined by a physical focusing of the ultrasonic transducer elements 24, for example, along the surface of a sphere but also by controlling a phase of the signals to each of the ultrasonic transducer elements 24, both factors being captured by the focus data 34.

The transducer elements 24 of the therapy transducer array 12 each individually communicate with a radiofrequency (RF) amplifier 40 allowing the power and phase of excitation of each of the transducer elements 24 to be controlled to produce the desired microbubbles at a desired location. The RF amplifier 40 and the other components described herein may be controlled by a computer 42, for example, including one or more processors and electronic memory holding a stored program 44 operating to implement a therapy session providing a repeated set of excitation pulses to generate cavitation over a treatment duration and to provide the focus data 34. The computer 42 may also include a program 45 to be discussed below implementing features of the present invention. For this purpose, the computer 42 may include a cursor control device 43 such a mouse or trackball or the like to identify locations on the display.

Histotripsy systems, including the above described components and suitable for use with the present invention, are commercially available from Histosonics, Inc of Minnesota under the trade name Edison and described, for example, in U.S. Pat. No. 1,1819,712, hereby incorporated by reference.

Referring still the FIG. 1, the present invention may employ a second independent ultrasound probe 50 which may, for example, be a handheld ultrasonic probe of conventional design or an intra-cavity probe such as a transesophageal echocardiogram probe (TEE) or an intracardiac echocardiography probe (ICE) or the like. Generally, the independent ultrasound probe 50 will be freely positionable with respect to the therapy transducer array 12 (unlike the ultrasound imaging probe 30) and will include a two-dimensional array of transmitting and receiving transducer elements 51 for generating backscatter B-mode images from ultrasonic signals transmitted from the probe 50 or ultrasound transmission tomography images from signals transmitted from the therapy transducer array 12. In practice, the probe 50 will be positioned to image the focal point 28 at a different range and/or angle than provided by the probe 30, permitting the probe 50 to avoid occluding structures such as bones, air cavities, or the like.

The probe 50 may communicate with a second image processor 52 developing a B-mode image data received by compositor 54 for ultimate display on display 58.

The probe 50 may also communicate with a localizer circuit 60 that will be used to determine the relative location of the probe 50 (orientation and displacement) with respect to the therapy transducer array 12 and hence with respect to the focal point 28 known in relationship to the position of the therapy transducer array 12. Generally, the localizer circuit 60 operates in conjunction with a program 45 on the computer 42 which, in one embodiment, may control the RF amplifier 40 to excite particular transducer elements 24 of the therapy transducer array 12 that are separated across the array. These excitations produce ranging signals that will be used to deduce a distance of separation between the transducer elements 24 of the therapy transducer array 12 and the transducer elements 51 of the probe 50, thus locating these two elements with respect to each other. The excitation is performed in such a way as to allow the signal from each transducer element 24 to be distinguished, for example, by separation in time, frequency modulation, or the like. The receipt of these signals at transducer elements 51 are monitored to determine the relative location and orientation of the probe 50 as will be discussed in more detail below.

After the localizer circuit 60 determines a location of the probe 50 with respect to the therapy transducer array 12, this information is provided to the compositor 54 which may use the focus data 34 to add a fiducial mark 38 to the B-mode image 56 presented on display 58 from the data of the probe 50. Alternatively, and as will also be described below, the known location of the probe 50 may be used to add a targeting mark 41 to the B-mode image 56 that will be used to steer the therapy transducer array 12 so that its focal point 28 per focus data 34 aligns with the targeting mark 41.

The distance derived from the ranging pulses in the localization process is dependent on the sound speed of the pulses through different tissues 13 of the patient 16. For this reason, the localizer circuit 60 may make sound speed corrections in calculating the location of the probe 50. The sound speed corrections may make use of the current location of the probe 50 and use either of the image from the image processor 52 or a set of independently obtained non-ultrasound image data 62, for example, from computed tomography or magnetic resonance imaging. These images, in turn, may be used to characterize the tissue traversed in the B-mode image with respect to sound speed for different tissue types to better assess sound speed of the ranging pulses. The localizer circuit 60 may also use other methods of correcting sound speed including through temperature measurements of the patient tissue or through an additional measurements of distances between transducers that over constrain the localization process allowing errors to be detected.

The localizer circuit 60 may also be used to fuse or combine the non-ultrasound image data 62 or the B-mode image 36 with the B-mode image 56 presented to the user to provide additional information or improved image quality. For example, non-ultrasound image data 62 may be used to accentuate tissue to be treated as identified with a different modality. In some embodiments, display 58 may also provide an orientation compass 64 generally indicating an orientation of that image with respect to the therapy transducer array 12. Alternatively, the B-mode image 56 may be locked into an orientation matching that of the image obtained from the ultrasound imaging probe 30 allowing it to be augmented with the B-mode image 36.

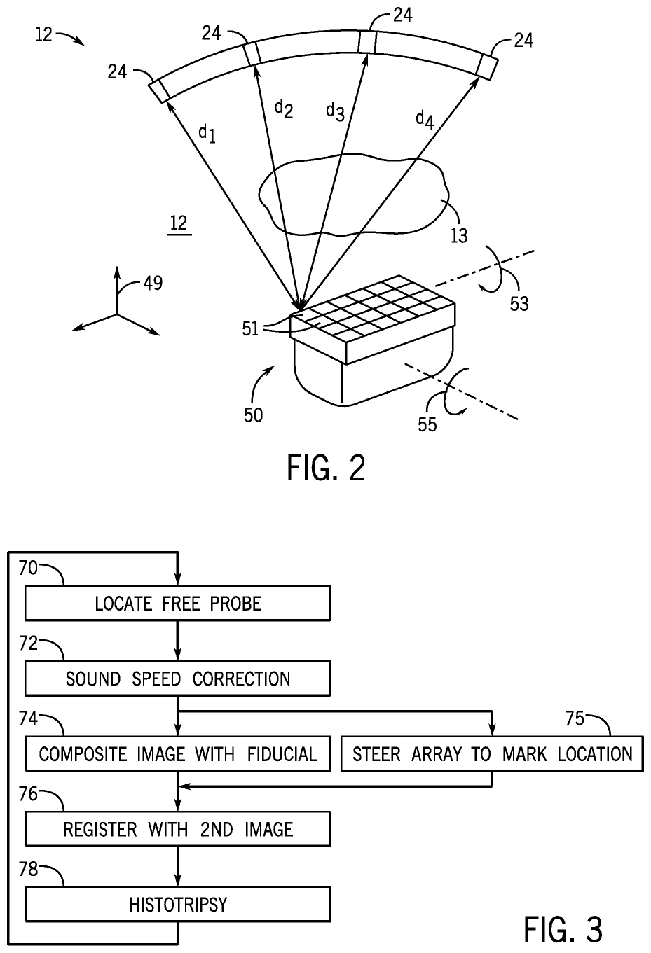

Referring now to FIGS. 2 and 3, the processes and components discussed above with respect to FIG. 1, including the programs 44 and 45 executed on the computer 42, may implement a histotripsy procedure on the patient 16, beginning as indicated by process block 70 by localization of the freely positionable probe 50. As noted above, in one embodiment, this localization may be performed by sequentially coupling a pulse from different spatially separated transducer elements 24 of the therapy transducer array 12 into the patient 16 and measuring the arrival time of each pulse at a different one of the transducer elements 51 of the probe 50. This time-of-flight may be used together with an assumption about sound speed to deduce a separation distance between each transducer element 24 and each selected transducer element 51. In one embodiment, the time-of-flight is determined by measuring a time after triggered by the RF amplifier 40 to a time where that pulse is received from the probe 50. Comparable information can be obtained without synchronizing the transmission and reception of pulses, however, simply by separating transmitted signals by a predetermined known time and analyzing differences between the receipt time of the signals at each transducer element 51.

Each of these different time-of-flight measurements and their corresponding imputed distances provide for a trilateralization or multi-lateralization of the location of each of the transducer elements 51. Generally, at least three independent distance measurements will be made for each transducer element 51 from different transducer elements 24. These independent distance measurements define a set of solutions being the intersection of three spheres having radii equal to the corresponding distances and centered about the respective transducer elements 24. The set of solutions includes two points, with one point being generally outside of the patient body and accordingly removed. Four or more distance measurements for each transducer element 24 remove this ambiguity or provide an over-constrained solution which may be useful for determining variations in sound speed as will be discussed.

By locating multiple separated transducer elements 24 in an array having known transducer element locations, the localizer circuit 60 may not only determine the location of each transducer element 51 in Cartesian space but also the orientation (yaw 53 and pitch 55) of the array of transducer elements 51. These measurements may be made repeatedly during histotripsy process, making use of the fact that the histotripsy signals are transmitted at a low duty cycle to accurately determine the field-of-view of the probe 50 with respect to the therapy transducer array 12.

At process block 72, each determination of the location of probe 50 may be used in an iterative sound speed correction refining the assumption of sound speed used for distance calculation. In one embodiment, the location information of the probe 50, and in particular the acquired image data or non-ultrasound image data 62 registered to the location of an orientation of the probe 50, may be used to characterize the tissue through which the sound passes. This characterization identifies tissues in the image and associates them with empirically determined sound speeds, for example, using a stored lookup table. Different tissue types may be determined by the quality of the image data of each voxel or through the use of organ segmentation, identifying organs from acquired image data. To the extent that the acquired distance data may over constrain the location problem, these differences may be used to identify sound speed anomalies, for example, to remove measurements that are inconsistent with other measurements or to affect a compromise with inconsistent measurements. Tissue temperature measurements and elastographic measurements may also be used to further characterize the tissue with respect to sound speed.

At process box 74, as noted above, the known location of the ultrasound probe 50 permits the compositing of a fiducial mark 38 indicating the focal point 28 on the B-mode image 56 obtained with the probe 50 to the extent that that focal point 28 would be within the plane of the developed image. This allows the operator to accurately assess whether the generated microbubbles are at the desired focal point and to make adjustments in the focal point appropriately, for example, to correct for aberrations. Such adjustments may also be implemented automatically from this data.

Alternatively or in addition, at process block 75, the known location of the ultrasound probe 50 with respect to the therapy transducer array 12 permits the user to place a targeting mark 41 on the B-mode image 56 using the cursor control device 43 that will also be registered with the therapy transducer 12. This target mark 41 may indicate a desired focal point 28 of the therapy transducer 12 and be used to realign the therapy transducer 12 through servo positioning motors 20 under the control of the computer 42 to move the actual focal point 28 to the targeting mark 41. This allows the operator to accurately manipulate the focal point 28 through the superior image obtained from the probe 50.

At process block 76, the known location of the probe 50 may be used to register and align data additional data from another source including from the B-mode image 36 or the non-ultrasound image data 62 with the field-of-view of the probe 50 using sensor fusion techniques generally understood in the art.

Finally, at process box 78, the RF amplifier 40 may be controlled by the program 44 to implement a given fraction of the treatment of the focal point 28 with microbubble cavitation.

These processes of process box 70-78 may then be repeated under control of program 44 for the duration of the treatment process during which the focal point 28 may be steered through to tumorous tissue to destroy the same.

While the localization of the probe 50 may most conveniently be done by using transducer elements 24 of the therapy transducer array 12 and transducer elements 51 of the probe 50, it will be generally understood that separate dedicated beacon transmitters and receivers may also be used located on the therapy transducer array 12 or in alternative locations provided they are fixed with respect to the therapy transducer array 12 or probe 50, respectively.

Certain terminology is used herein for purposes of reference only, and thus is not intended to be limiting., for example,, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to a circuit, a processor, or a computer should be understood to broadly include electronic circuits for implementing the indicated functions including discrete circuitry, specialized integrated circuits, programmable computers or microprocessors, microcontrollers or FPGAs and the like. Such circuits may be implemented in a single device or distributed set of components communicating over a network or the like. References to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An ultrasound therapy system, comprising;
an ultrasound therapy transducer array adapted to deliver histotripsy pulses into a subject to generate cavitation in the subject at a focal region;
an ultrasound probe independently positionable with respect to the ultrasound therapy transducer array and adapted to receive acoustic signals from the subject for producing an ultrasonic image of the subject;
a first set of ultrasonic transducer elements at predetermined separated locations fixed with respect to the ultrasonic therapy transducer array and adapted to couple acoustic signals between the first set of ultrasonic transducer elements and the subject;
a second set of ultrasonic transducer elements at predetermined separated locations fixed with respect to the ultrasound probe and adapted to couple acoustic signals between the second set of ultrasonic transducer elements and the subject; and
a localizing circuit operating to activate transducers of one of the first or second set of ultrasonic transducers to couple independently distinguishable locating acoustic signals into the subject and to monitor a receipt of the independently distinguishable locating acoustic signals at transducers of a different of the first and second set of ultrasonic transducers to deduce a relative location of the ultrasonic probe with respect to the ultrasound therapy transducer.

2. The ultrasound therapy system of claim 1 wherein the second set of ultrasonic transducers is separated in two dimensions and the localizing circuit uniquely localizes the ultrasonic probe with respect to relative translation and rotation of the ultrasonic probe with respect to the ultrasonic therapy transducer.

3. The ultrasound therapy system of claim 1 further including at least one image processing circuit communicating with the ultrasound probe to produce an ultrasonic image of the subject from ultrasonic signals received by the ultrasound probe;
a compositor communicating with the localizing circuit to register a fiducial mark representing the focal region with respect to the ultrasonic image; and
a display communicating with the compositor to display an image of the subject registered to the fiducial mark.

4. The ultrasound therapy system of claim 1 wherein the therapy transducer array further includes at least one image processing circuit communicating with the ultrasound probe to produce an ultrasonic image of the subject from ultrasonic signals received by the ultrasound probe;
a cursor control device communicating with the localizing circuit to place a targeting mark representing a desired treatment area on the ultrasonic image; and
a motorized actuator communicating with the therapy transducer array and operating to steer the focal region into alignment with the targeting mark with respect to the subject.

5. The ultrasound therapy system of claim 1 wherein the ultrasound probe transmits and receives ultrasonic signals for a production of the ultrasonic image of the subject.

6. The ultrasound therapy system of claim 1 further including a second ultrasound probe fixed with respect to the ultrasound therapy transducer and communicating with the at least one image processing circuit to develop a second ultrasound image of the subject from ultrasound signals received by the second ultrasound probe.

7. The ultrasound therapy system of claim 1 further including a computer memory holding non-ultrasound image data of the subject obtained from a non-ultrasonic imaging modality and wherein the compositor registers the non-ultrasound image data to the ultrasonic image to display on the display a composite of the ultrasound image and the non-ultrasound image data.

8. The ultrasound therapy system of claim 1 wherein the first set of ultrasonic transducer elements provides at least three ultrasonic transducer elements.

9. The ultrasound therapy system of claim 1 wherein first set of ultrasonic transducer elements operates alternatively to provide the independently distinguishable locating acoustic signals and histotripsy pulses.

10. The ultrasound therapy system of claim 1 wherein the localizing circuit deduces the relative location of the different of the first and second ultrasonic transducer elements based on a speed of sound through the subject between the first and second set of ultrasonic transducers, and further including a sound speed corrector iteratively correcting an assumed sound speed using the deduced relative location of the ultrasonic probe with respect to the ultrasonic therapy transducer.

11. The ultrasound therapy system of claim 9 wherein the sound speed corrector iteratively corrects an assumed sound speed using at least one of image data obtained from the ultrasound probe and image data obtained from a non-ultrasound imaging modality.

12. A method of an ultrasound therapy system using:
an ultrasound therapy transducer array adapted to deliver histotripsy pulses into a subject to generate cavitation in the subject at a focal region;
an ultrasound probe independently positionable with respect to the ultrasound therapy transducer array and adapted to receive acoustic signals from the subject for producing an ultrasonic image of the subject;
a first set of ultrasonic transducer elements at predetermined separated locations fixed with respect to the ultrasonic therapy transducer array and adapted to couple acoustic signals between the first set of ultrasonic transducer elements and the subject;
a second set of ultrasonic transducer elements at predetermined separated locations fixed with respect to the ultrasound probe and adapted to couple acoustic signals between the second set of ultrasonic transducer elements and the subject; and
a localizing circuit operating to activate transducers of one of the first or second set of ultrasonic transducers to couple independently distinguishable locating acoustic signals into the subject and to monitor a receipt of the independently distinguishable locating acoustic signals at transducers of a different of the first and second set of ultrasonic transducers to deduce a relative location of the ultrasonic probe with respect to the ultrasound therapy transducer, the method comprising:
(a) activating transducers of one of the first or second set of ultrasonic transducers to send independently distinguishable locating acoustic signals into the subject;
(b) monitor a receipt of the independently distinguishable locating acoustic signals at transducers of a different of the first and second set of ultrasonic transducers to deduce a relative location of the ultrasonic probe with respect to the ultrasound therapy transducer.

13. The method of claim 12 wherein the second set of ultrasonic transducers is separated in two dimensions and the localizing circuit uniquely localizes the ultrasonic probe with respect to relative translation and rotation of the ultrasonic probe with respect to the ultrasonic therapy transducer.

14. The method of claim 12 further including at least one image processing circuit communicating with the ultrasound probe to produce an ultrasonic image of the subject from ultrasonic signals received by the ultrasound probe and including:
(c) registering a fiducial mark representing the focal region with respect to the ultrasonic image; and
(d) displaying an image of the subject registered to the fiducial mark.

15. The method of claim 12 further including at least one image processing circuit communicating with the ultrasound probe to produce an ultrasonic image of the subject from ultrasonic signals received by the ultrasound probe and including;
communicating with the localizing circuit to place a targeting mark representing a desired treatment area on the ultrasonic image; and
operating a motorized actuator communicating with the therapy transducer array and operating to steer the focal region into alignment with the targeting mark with respect to the subject.

16. The method of claim 12 further including registering non-ultrasound image data to the ultrasonic image to display on the display a composite of the ultrasound image and the non-ultrasound image data.

17. The method of claim 12 wherein first set of ultrasonic transducer elements operates alternatively to provide the independently distinguishable locating acoustic signals and histotripsy pulses.

18. The method of claim 12 wherein a deduction of the relative location of the different of the first and second ultrasonic transducer elements is based on a speed of sound through the subject between the first and second set of ultrasonic transducers, and further including an iteratively correcting and assumed sound speed using the deduced relative location of the ultrasonic probe with respect to the ultrasonic therapy transducer.

19. The method of claim 18 wherein the assumed sound speed is corrected using at least one of image data obtained from the ultrasound probe and image data obtained from a non-ultrasound imaging modality.

20. The method of claim 12 wherein the ultrasound probe is placed within a cavity within the subject.
